(19) United States
(12) Patent Application Publication
(10) Pub. No.: US 2002/0004604 A1
(43) Pub. Date: Jan. 10, 2002

(54) Method for the preparation of citalopram

(75) Inventors: Hans Petersen, Vanlose (DK); Michael Harold Rock, Hvidovre (DK)

Correspondence Address: DARBY & DARBY P.C., 805 Third Avenue, New York, NY, 10022, US

(73) Assignee: H. Lundbeck A/S, Copenhagen (DK)

(21) Appl. No.: 09/794,755

(22) Filed: Feb. 26, 2001

(30) Foreign Application Priority Data
Feb. 24, 2000 (DK) PA 2000 00296

(51) Int. Cl. C07D 307/87

(52) U.S. Cl. 549/462

(57) ABSTRACT

A method for the preparation of citalopram wherein the aldehyde of formula

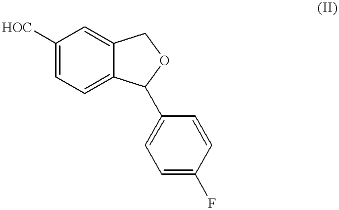

is converted to the corresponding 5-cyano compound of formula (I)

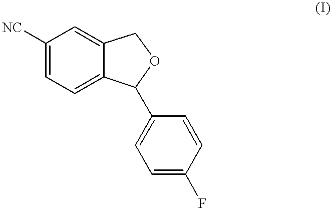

which is alkylated to form citalopram, which is isolated in the form of the base or an acid addition salt thereof.

The present invention relates to a method for the preparation of the well-known antidepressant drug citalopram, 1-[3-(dimethylamino)propyl]-1-(4-fluorophenyl)-1,3-dihydro-5-isobenzofuran-carbonitrile.

### BACKGROUND OF THE INVENTION

Citalopram is a well-known antidepressant drug that has now been on the market for some years and has the following structure:

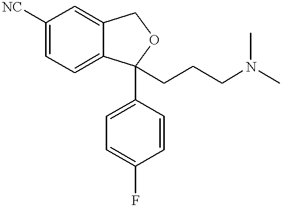

It is a selective, centrally acting serotonin (5-hydroxytryptamine; 5-HT) reuptake inhibitor, accordingly having antidepressant activities. The antidepressant activity of the compound has been reported in several publications, eg. J. Hyttel Prog. Neuro-Psychopharmacol. & Biol. Psychiat. 1982, 6, 277-295 and A. Gravem Acta Psychiatr. Scand. 1987, 75, 478-486. The compound has further been disclosed to show effects in the treatment of dementia and cerebrovascular disorders, EP-A-474580.

Citalopram was first disclosed in DE 2,657,013, corresponding to U.S. Pat. No. 4,136,193. This patent publication describes the preparation of citalopram by one method and outlines a further method which may be used for preparing citalopram.

According to the process described, the corresponding 1-(4-fluorophenyl)-1,3-dihydro-5-isobenzofurancarbonitrile is reacted with 3-(N,N-dimethylamino)propyl-chloride in the presence of methylsulfinylmethide as condensing agent. The starting material was prepared from the corresponding 5-bromo derivative by reaction with cuprous cyanide.

International patent application No. WO 98/019511 discloses a process for the manufacture of citalopram wherein a (4-(cyano, alkyloxycarbonyl or alkylaminocarbonyl)-2-hydroxymethylphenyl-(4-fluorophenyl)methanol compound is subjected to ring closure. The resulting 5-(alkyloxycarbonyl or alkylaminocarbonyl)-1-(4-fluorophenyl)-1,3-dihydroisobenzofuran is converted to the corresponding 5-cyano derivative and the 5-cyano derivative is then alkylated with a (3-dimethylamino)propylhalogenide in order to obtain citalopram.

It has now, surprisingly, been found that citalopram may be manufactured by a novel favourable process via 1-(4-fluorophenyl)-1,3-dihydroisobenzofurane-5-formaldehyde prepared by ring closure of 2,4-dihydroxymethyl-1-[1-(4-fluorophenyl)-1-hydroxy-1-methyl]benzene and oxidation of the resulting 5-hydroxymethyl-1-(4-fluorophenyl)-1,3-duhydroisobenzofuran.

### SUMMARY OF THE INVENTION

The present invention thus relates to a method for the preparation of citalopram wherein the aldehyde of formula

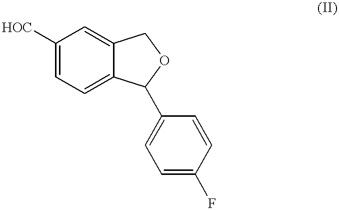

is converted to the corresponding 5-cyano compound of formula (I)

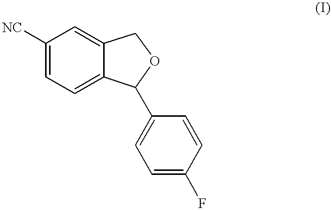

followed by alkylation to form citalopram, which is isolated in the form of the base or as a pharmaceutically acceptable acid addition salt thereof.

In a particularly preferred embodiment of the invention, the compound of formula (II) is prepared by reduction of a compound of formula

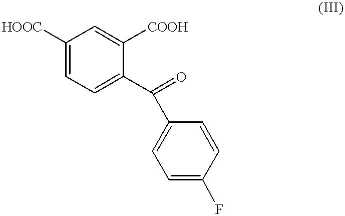

to form a compound of formula

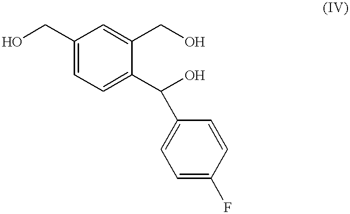

followed by ring closure to form a compound having the formula

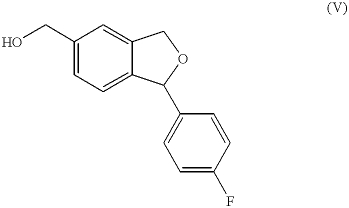

which, is then oxidised to form the compound of formula (II).

The invention also relates to the intermediate having the formula

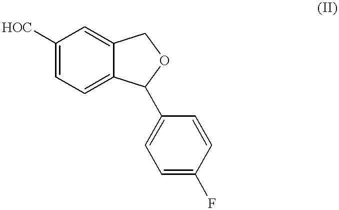

or a salt thereof.

Finally, the invention relates to an antidepressant pharmaceutical composition comprising citalopram manufactured by a process of the invention.

According to a preferred embodiment of the invention, the alkylation is carried out by reaction of a compound of formula (1) with a 3-(dimethylamino)propylhalogenide as described in U.S. Pat. No. 4,136,193.

### DETAILED DESCRIPTION OF THE INVENTION

According to the present invention, the citalopram intermediates having the formulas (I) and (II) may be prepared by the process illustrated in the following reaction scheme:

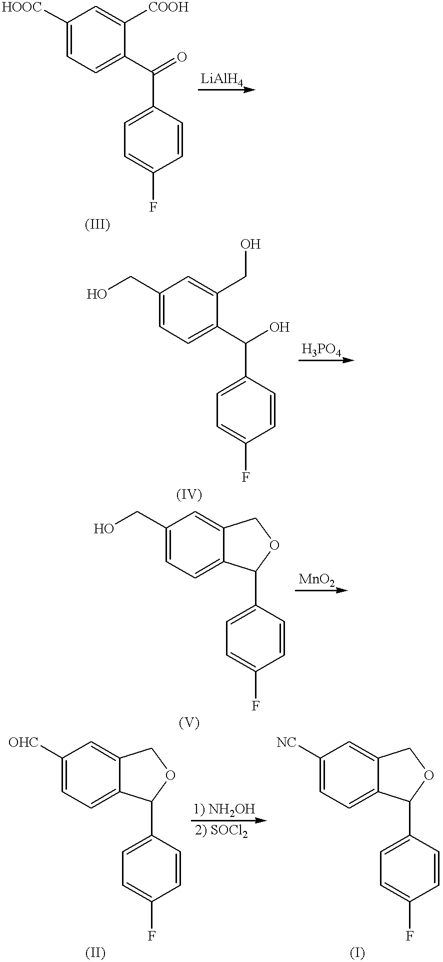

The conversion of the compound of formula (III) to a compound of formula (V) may be carried out using conventional techniques. Thus, the reducing agent used for reduction of the compound of (III) may be LiAlH₄, NaAlH₂(OCH₂CH₂OMe)₂, NaBH₄/BF₃.Et₂O, NaBH₄/I₂ or any another suitable reducing agent, the ring closure of the compound of formula (IV) may be carried out by dehydration using mineral acids such as H₃PO₄, H₂SO₄, HCl or another suitable dehydrating agent or by ring closure of the corresponding active ester in presence of a base as described in EP 347 066. The oxidation of the compound of formula (V) may be carried out using MnO₂, NiO₂, (NH₄)₂Ce(NO₃)₆ or another suitable oxidixing agent.

Conversion of the formaldehyde group of the compound of formula (II) to a cyano group may be carried out by reaction with hydroxylamine followed by treatment with a dehydrating agent such as SOCl₂. Other methods are described in WO 99/30548, see in particular page 6.

The compound of formula (III) may be prepared by oxidation of the corresponding dimethyl compound as described in N. S. Dokunikhin, B. V. Salov, A. S. Glagoleva Zhurnal Obshchei Khimii 1964, 34, 995-998.

The alkylation of the compound of formula (I) to form citalopram may be performed according to the process of U.S. Pat. No. 4,136,193 or WO 98/019611.

Alternatively, the alkylation may be carried our as described in co-pending DK application No PA 200000353.

According to this process, citalopram is prepared by alkylation of a compound of formula (I) with a compound having the formula

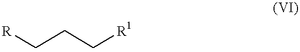

wherein R is halogen or —O—SO₂—X wherein X is alkyl, aryl, aralkyl or alkylaryl and R¹ is dimethylamino, —O—SO₂—X wherein X is alkyl, aryl, aralkyl or alkylaryl, or halogen; provided that R is not halogen when R¹ is dimethylamino, followed by isolation of citalopram where R is dimethylamino, or followed by reaction of the resulting compound of formula

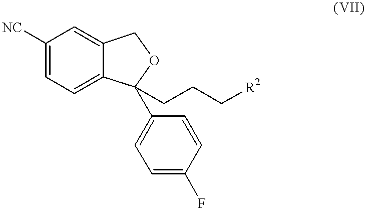

wherein R² is halogen or a group of formula —O—SO₂—X wherein X is as defined above with dimethylamin or a metal salt thereof; and thereafter isolation of citalopram or a pharmaceutically acceptable acid addition salt thereof.

The alkylation step where the compound of formula (I) is reacted with a compound of formula (VI) is suitably carried out by treatment of the compound of formula (I) with a base such as for example LDA (lithium diisopropylamine), LiHMDS (lithium hexamethyldisilazane), NaH, NaHMDS (sodium hexamethyldisilazane), or NaOMe in an aprotic organic solvent such as THF (tetrahydrofurane), DMF (dimethylformamide), NMP (N-methylpyrrolidon), ethers such as diethylether, or dioxalane, toluene, benzene, or alkanes and mixtures thereof. The anion formed is then reacted with a compound of formula (VI) whereby a group of formula —CH₂—CH₂—CH₂—R² or a group of formula —CH₂—CH₂—CH₂—N(CH₃)₂ is introduced into position 1 of the isobenzofuranyl ring system.

The compound of formula (VII) is then reacted with dimethylamin or a metal salt thereof, such as M⁺, ⁻N(CH₃)₂ wherein M⁺ is Li⁺ or Na⁺. The reaction is suitably carried out in an aprotic organic solvent such as THF (tetrahydrofurane), DMF (dimethylformamide), NMP (N-methyl pyrrolidon), ethers such as diethylether, or dioxalane, toluene, benzene, or alkanes and mixtures thereof.

The reaction conditions, solvents, etc. used for the reactions described above are conventional conditions for such reactions and may easily be determined by a person skilled in the art.

Other methods for the alkylation of a compound of formula (I) to form citalopram are described in co-pending DK application No 200000404.

According to the processes described herein, citalopram may be prepared by:

a) Reaction of a compound of formula (I) with a compound of formula HCO—(CH₂)₂—N(CH₃)₂ followed by dehydration to form a compound of formula (VIII)

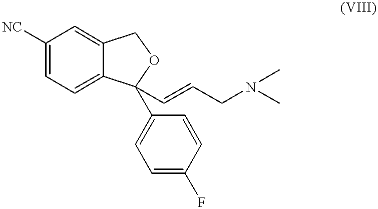

and reduction of the compound of formula (VIII) to form citalopram;

b) Reaction of a compound of formula (I) with a compound of formula

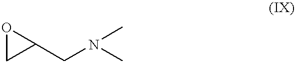

followed by dehydration to form a compound of formula (VIII) as above and reduction to form citalopram; or

c) Reaction of a compound of formula (I) with a compound of formula Y—CH₂—CH═CH₂ wherein Y is a suitable leaving group to form a compound of formula

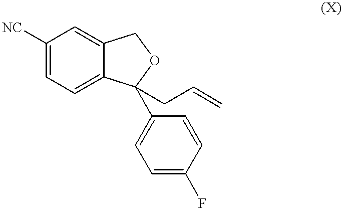

followed by peroxidation of the double bond and reaction with dimethyl amine to form a compound of formula (VIII) and reduction of the compound of formula (VIII) to form citalopram.

The alkylation step where the compound of formula (I) with a compound of formula HCO—(CH₂)₂—N(CH₃)₂, Y—CH₂—CH═CH₂, or of formula (IX) is suitably carried out as described above for the reaction of a compound of formula (I) with a compound of formula (VI).

Other methods for alkylation of a compound of formula (I) to form citalopram are described in co-pending DK applications Nos PA 200000401, PA 200000403, PA 200000404, PA 200000414 and PA 200000415.

Citalopram is on the market as an antidepressant drug in the form of the racemate. However, in the near future the active S-enantiomer of citalopram is also going to be introduced to the market.

S-citalopram may be prepared by separation of the optically active isomers by chromatography.

Throughout the specification and claims, the term alkyl refers to a branched or unbranched alkyl group having from one to six carbon atoms inclusive, such as methyl, ethyl, 1-propyl, 2-propyl, 1-butyl, 2-butyl, 2-methyl-2-propyl, 2,2-dimethyl-1-ethyl and 2-methyl-1-propyl.

The term aryl refers to a mono- or bicyclic carbocyclic aromatic group, such as phenyl and naphthyl, in particular phenyl.

The term aralkyl refers to aryl-alkyl, wherein aryl and alkyl is as defined above.

Halogen means chloro, bromo or iodo.

Citalopram may be used as the free base or as a pharmaceutically acceptable acid addition salt thereof. As acid addition salts, such salts formed with organic or inorganic acids may be used. Exemplary of such organic salts are those with maleic, fumaric, benzoic, ascorbic, succinic, oxalic, bismethylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzene sulfonic and theophylline acetic acids, as well as the 8-halotheophyllines, for example 8-bromotheophylline. Exemplary of such inorganic salts are those with hydrochloric, hydrobromic, sulfuric, sulfamic, phosphoric and nitric acids.

The acid addition salts of the compounds may be prepared by methods known in the art. The base is reacted with either the calculated amount of acid in a water miscible solvent, such as acetone or ethanol, with subsequent isolation of the salt by concentration and cooling, or with an excess of the acid in a water immiscible solvent, such as ethylether, ethylacetate or dichloromethane, with the salt separating spontaneously.

The pharmaceutical compositions of the invention may be administered in any suitable way and in any suitable form, for example orally in the form of tablets, capsules, powders or syrups or parenterally in the form of usual sterile solutions for injection.

The pharmaceutical formulations of the invention may be prepared by conventional methods in the art. For example, tablets may be prepared by mixing the active ingredient with ordinary adjuvants and/or diluents and subsequently compressing the mixture in a conventional tabletting maschine. Examples of adjuvants or diluents comprise: Corn starch, potato starch, talcum, magnesium stearate, gelatine, lactose, gums, and the like. Any other adjuvant or additive, colourings, aroma, preservatives etc. may be used provided that they are compatible with the active ingredients.

Solutions for injections may be prepared by solving the active ingredient and possible additives in a part of the solvent for injection, preferably sterile water, adjusting the solution to the desired volume, sterilising the solution and filling it in suitable ampoules or vials. Any suitable additive conventionally used in the art may be added, such as tonicity agents, preservatives, antioxidants, etc.

The invention is further illustrated by the following examples.

### EXAMPLE 1

1-(4-fluorophenyl)-1,3-dihydroisobenzofuran-5-carbonitrile

Step 1: 2,5-Dihydroxymethyl-1-[1-(4-fluoro-phenyl)-1-hydroxy-1-methyl]benzene LiAlH₄ (15.2 g, 0.6 mole) is covered with toluene (800 mL). THF (400 mL) is added. 4-Fluorobenzophenone-2′, 4′-dicarboxylic acid¹) (58 g, 0.2 mole) is added in portions of about 10 grams. The temperature is allowed to rise to 50° C. The mixture is heated at reflux temperature for 1½ hour. After cooling to 10° C., water (100 mL) is added carefully. K₂CO₃ (150 g) is added and the suspension is stirred for ½ hour. After filtration the volatiles are evaporated off in vacuo. Yield (50 g, 95%). The title compound is obtained as an oil. ¹H NMR (DMSO-d₆, 500 MHz): 4.28 (2H, s), 4.41 (2H, s), 5.75 (1H, s), 6.95-7.35 (7H).

Step 2: 5-Hydroxymethyl-1-(4-fluorophenyl)-I, 3-dihydroisobenzofurane. H₃PO₄ (200 mL, 60%) is added to triol 2,4-dihydroxymethyl-1-[1-(4-fluorophenyl)-1-hydroxy-1-methyl]-benzene (50 g) and the mixture is heated to 80° C. for 2 hours. On cooling, the title compound crystallises and is filtered off. Recrystallization from EtOH/water ((1:3), 400 mL). Yield: 44 grams (90%, total for step 1 and 2). Mp: 101-03 C. ¹H NMR (DMSO-d₆, 500 MHz): 4.51 (2H, s), 5.08 (1H, d J=12.5 Hz), 5.26 (1H, d J=12.5 Hz), 6.14 (1H, s), 6.96-7.4 (7H).

Step 3: 1-(4-Fluorophenyl)-1,3-dihydroisobenzofurane-5-formaldehyde.

The hydroxymethyl phthalan 5-hydroxymethyl-1-(4-fluorophenyl)-1,3-dihydroisobenzofurane (24 grams, 0.1 mole) is dissolved in DCM (500 mL). MnO₂ (52 grams) is added in three portions. The mixture is stirred for 16 hours at room temperature. After filtration using a pad of filter help and silica the solvent is evaporated off in vacuo and the title compound is obtained as an oil. Yield: 24 g (100%). ¹H NMR (CDCl₃, 500 MHz): 5.22 (1H, d J=12.5 Hz), 5.36 (1H, d J=12.5 Hz), 6.15 (1H, s), 7.0-7.73 (7H), 10.00 (1H,s).

Step 4: 1-(4-Fluorophenyl)-1,3-dihydroisobenzofurane-5-carbonitrile.

To aldehyde 1-(4-fluorophenyl)-1,3-dihydroisobenzofurane-5-formaldehyde (2.4 grams, 0.01 mole) dissolved in EtOH (10 mL) is added NH₂OH,HCl (1 gram, 0.015 mole) and NaOH (0.6 gram, 0.015 mole) dissolved in water (25 mL). The mixture is heated at reflux temperature for ½ hour. After cooling to room temperature, the reaction mixture is left for 2 hour. The crystals are filtered off and washed with cold water (2×10 mL) and dried. The oxime is suspended in toluene (10 mL) and SOCl₂ (1.3 mL) is added. The mixture is heated to 80° C. for 1 hour. After cooling, the volatiles are evaporated off in vacuo and the title compound is crystallized from heptane. Yield: 2.0 gram (84%) DSC (onset): 98 C.

¹⁾ N. S. Dokunikhin, B. V. Salov, A. S. Glagoleva Zhurnal Obshchei Khimii 1964, 34, 995-998.

1. A method for the preparation of citalopram wherein the aldehyde of formula 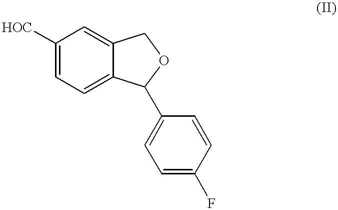
is converted to the corresponding 5-cyano compound of formula (I) 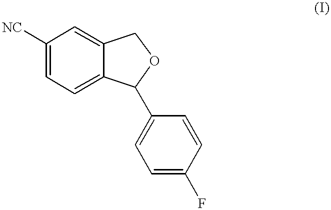
which is alkylated to form citalopram, which is isolated in the form of the base or an acid addition salt thereof.

2. The method according to claim 1 wherein the compound of formula (II) is prepared by reduction of a compound of formula 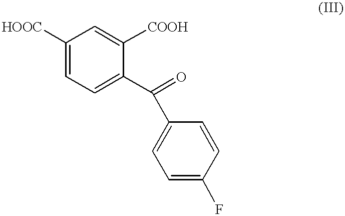
to form a compound of formula 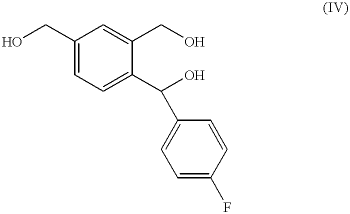
followed by ring closure to form a compound having the formula 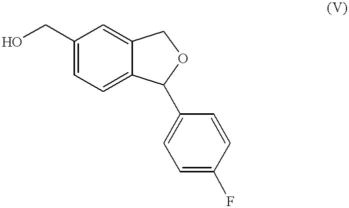
which is then oxidised to form the compound of formula (II)

3. The method of claim 1 wherein the alkylation is made by reaction of the compound of formula I with a 3-(dimethyl amino)propyl halogenide.

4. A compound having the formula 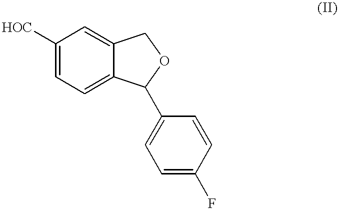
or an acid addition salt thereof.

5. An antidepressant pharmaceutical composition comprising citalopram manufactured by the process of any of claims 1 to 3.
